(19) United States
(12) Patent Application Publication
(10) Pub. No.: US 2022/0241620 A1
(43) Pub. Date: Aug. 4, 2022

(54) PROTECTIVE MASK

(71) Applicant: EASTERN MARK ENTERPRISES LIMITED, TAIPEI (TW)

(72) Inventor: Chiu-Chun LIN, TAIPEI (TW)

(21) Appl. No.: 17/166,222

(22) Filed: Feb. 3, 2021

(51) Int. Cl. A62B 18/04; A41D 13/11; A61F 9/02; A62B 7/10; A62B 23/02; A62B 18/08

(52) U.S. Cl. CPC A62B 18/045; A41D 13/1153; A41D 13/1184; A62B 18/084; A62B 7/10; A62B 23/02; A61F 9/028

(57) ABSTRACT

Provided is a protective mask, including: a mask, a hood, a barrier layer and an air guide, the mask comprising protective goggles, air outlet and air inlet, the air outlet and air inlet being disposed respectively at the upper and lower ends of the protective goggles, the air inlet being disposed with a filter, the air inlet and the air outlet communicating with the inside of the mask; the hood being connected to the mask, and covering the edge of the mask; the barrier layer being disposed at the connection between the mask and the hood to block external air from entering; and the air guide being disposed at the air outlet. The air enters the mask and the hood through the air inlet, guided by the air guide to the air outlet, and then discharged to the outside through the air outlet.

### BACKGROUND OF THE INVENTION

### 1. Field of the Invention

The present invention relates generally to a mask structure, and more particularly, to a protective mask with an improved structure.

### 2. The Prior Arts

In various working environments, there may exist different types of risk factors, such as airborne droplets, pollutants, pathogens, pollen, and floating fine asbestos fiber dust, and the inhalation or direct contact with the aforementioned risk factors without barriers will cause serious injury. So, workers need to wear protective clothing to avoid direct exposure to risk factors.

Moreover, in special professional venues, such as semiconductor production, biochemical technology, precision machinery, pharmaceuticals, and hospitals and other industries that are particularly sensitive to environmental pollution, environmental parameters must be controlled within a certain required range to avoid affecting the manufacturing process. For instance, the semiconductor industry has strict requirements on indoor temperature, humidity and cleanliness. To meet the cleanliness criteria, human hair, dander, sweat, and droplets are not allowed to be present in the working environment; therefore, it is necessary to block the factors affecting cleanliness by wearing special clothing.

On the other hand, protective clothing such as cleanroom clothing and anti-static clothing commonly used at present are usually integrated into a form to cover the whole body, exposing only the face, hands and feet, and then a facial mask, a hood, gloves or shoe covers and other accessory protective accessories are worn to achieve full-body protection effect.

Since the human body is actually in a large-area isolation state in the aforementioned situations, in addition to the risk factors or human pollutants, the heat and moisture emitted by the human body will also be trapped in the protective clothing. As such, the human body heat and moisture generated during work cannot be effectively dissipated, and the staff must wear protective clothing for a period of time in a very stuffy working environment, which will not only reduce work efficiency, but also greatly shorten the working time that the staff can withstand.

Although the temperature in cleanrooms in the high-tech field is mostly controlled by air conditioning and falls in the range of 25 degrees to 27 degrees Celsius, and in hospitals, laboratories and other places, there are also air conditioners to maintain the temperature, the human body will still feel quite stuffy and uncomfortable even with air conditioning when wearing protective clothing or cleanroom clothing.

Moreover, most of the working environment does not have air conditioning. In occasions such as chemical fiber or glass fiber processing, high-pressure cleaning, oil tank cleaning or painting treatment, and so on, wearing protective clothing to resist the risk factors of the external working environment with the extreme sultry feeling will increase the psychological and physical burden on the user.

Due to the extremely stuffy feeling caused by wearing protective clothing, most of the manufacturers have modified the material of the protective clothing, hoping to continuously improve the air permeability of the material to allow excessive heat and moisture to be dissipated.

However, the improved air permeability of the material has limits, which also cause the conflict between the objectives of insulation protection and ventilation and heat dissipation. Protective clothing made of high air permeability materials can only achieve so much in heat dissipation. Moreover, the unit price of high-permeability materials is expensive, which in turn increases the price of protective clothing and further reduces the willingness for manufacturers that require a large number of employees to wear protective clothing to work, or only need to wear protective clothing in a few specific events. In other words, high-priced protective clothing will become a considerable production cost, or will be limited by the number of uses.

Therefore, observing the aforementioned deficiencies has propelled the creation of the present invention.

### SUMMARY OF THE INVENTION

A primary objective of the present invention is to provide a protective mask with the arrangement of an air outlet and an air inlet, and with the arrangement of filters and air guides. The air enters the inside of the mask and the hood through the air inlet, the air guide is used to guide the air inside the mask and the hood to the air outlet, and the air is discharged to the outside of the mask and the hood through the air outlet. A barrier layer is used to block the outside air from entering the inside of the mask and the hood through the joint between the mask and the hood, so that the present invention can reduce the stuffiness inside the protective clothing and further improve the work efficiency, as well as greatly increase the work time the wearer can withstand.

To achieve the aforementioned objective, a protective mask provided by the present invention includes: a mask, including at least a pair of protective goggles, at least one air outlet and at least one air inlet, the air outlet being disposed at the upper end of the protective goggles, the air inlet being disposed at the lower end of the protective goggles, the air inlet being disposed with a filter, both the air inlet and the air outlet communicating with the inside of the mask; a hood, connected to the mask, and covering the edge of the mask; at least one barrier layer, disposed at the connection between the mask and the hood; and at least one air guide, disposed at the air outlet, wherein air entering the mask and the hood through the air inlet, the air inside the mask and the hood being guided by the air guide to the air outlet, and then discharged to the outside of the mask and the hood through the air outlet, and the barrier layer blocking external air from entering through the connection between the mask and the hood into the mask and the hood.

Preferably, the filter comprises a filtering material for filtering harmful substances in the air.

Preferably, the air guide is a fan, and the air guide drives the air to flow inside the mask and the hood.

Preferably, the number of the air inlets and the number of the filters are both two, with each air inlet disposed with a filter.

Preferably, the back of the hood is provided with at least one adjustable element, and the adjustable element is used to adjust the tightness of the hood.

Preferably, the chin portion of the hood is provided with at least one adjustable element, and the adjustable element is used to adjust the tightness of the hood.

Preferably, an opening is disposed in the chin portion of the hood in a recessed manner, and the opening is connected to an inner bag, and the inner bag has an accommodating space.

Preferably, the inner bag is located inside the mask and the hood, and the inner bag covers the air inlet.

Preferably, a zipper is provided at the edge of the opening, and the zipper is used to open or close the opening.

Preferably, at least one filtering material is further disposed in the accommodating space of the inner bag.

The protective mask provided by the present invention mainly has the arrangement of the air outlet and the air inlet, and the arrangement of the filters and the air guides. The air enters the inside of the mask and the hood through the air inlet, the air guide is used to guide the air inside the mask and the hood to the air outlet, and the air is discharged to the outside of the mask and the hood through the air outlet. A barrier layer is used to block the outside air from entering the inside of the mask and the hood through the joint between the mask and the hood to block the external polluted air, impurities and dust, so that the present invention can reduce the stuffiness inside the protective clothing and further improve the work efficiency, as well as greatly increase the work time the wearer can withstand.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a perspective view of the first embodiment of the present invention:

FIG. 2 is a schematic cross-sectional view taken along line A-A in FIG. 1;

FIG. 3 is a perspective view of wearing the first embodiment of the present invention;

FIG. 4 is a schematic view of the use state of the first embodiment of the present invention;

FIG. 5 is a perspective view of the second embodiment of the present invention:

FIG. 6 is a perspective view of the third embodiment of the present invention:

FIG. 7 is a perspective view of the fourth embodiment of the present invention; and

FIG. 8 is a schematic cross-sectional view taken along line A-A in FIG. 7.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

### First Embodiment

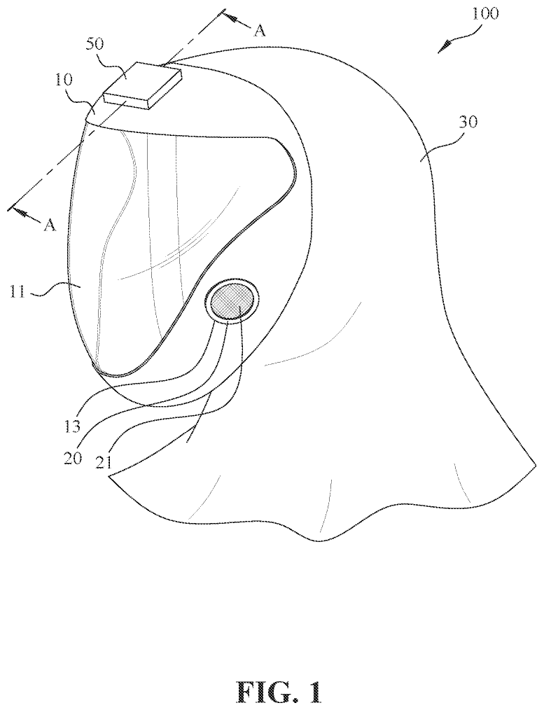

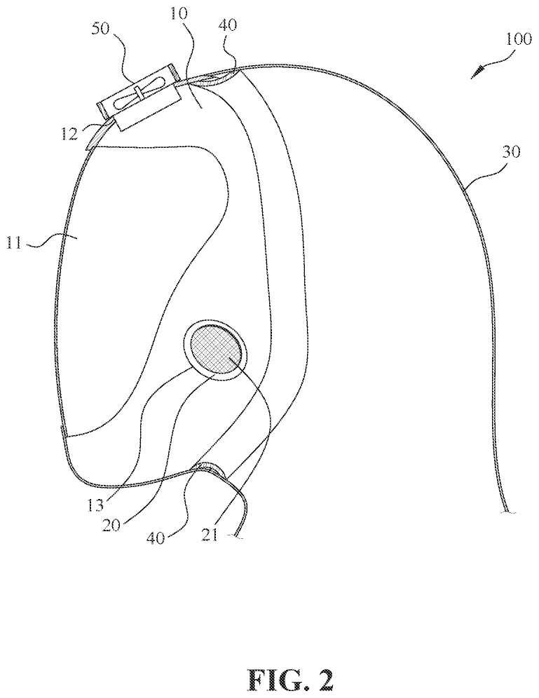

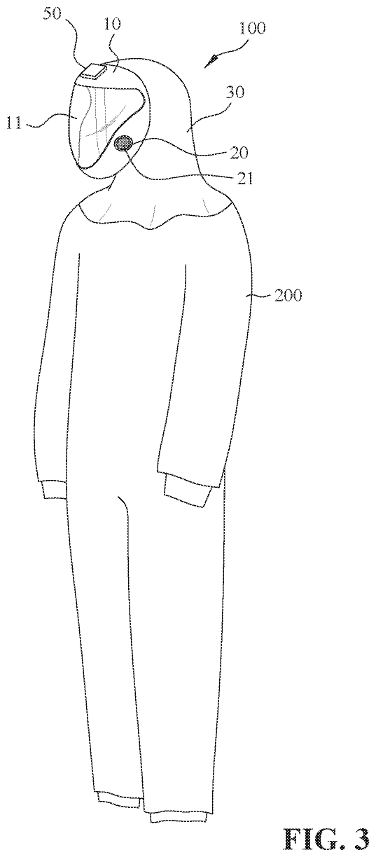

Referring to FIGS. 1-3, FIG. 1 is a perspective view of the first embodiment of the present invention; FIG. 2 is a schematic cross-sectional view taken along line A-A in FIG. 1; and FIG. 3 is a perspective view of wearing the first embodiment of the present invention. The present invention provides a protective mask 100, comprising: a mask 10, a hood 30, at least one barrier layer 40, and at least one air guide 50.

The mask 10 comprises at least a pair of protective goggles 11, at least one air outlet 12 and at least one air inlet 13. The air outlet 12 is disposed at the upper end of the protective goggles 11, the air inlet 13 is disposed at the lower end of the protective goggles 11, the air inlet 13 is disposed with a filter 20, both the air inlet 13 and the air outlet 12 communicates with the inside of the mask 10. In the present embodiment, as shown in FIG. 2, the filter 20 comprises a filtering material 21 for filtering harmful substances in the air. In the present embodiment, the protective goggles 11 are anti-fog and anti-glare protective goggles conforming to optical angle.

The hood 30 is connected to the mask 10, and covers the edge of the mask 10.

The at least one barrier layer 40 is disposed at the connection between the mask 10 and the hood 30.

The at least one air guide 50 is disposed at the air outlet 12. In the present embodiment, as shown in FIG. 2, the air guide 50 is a fan, and the air guide 50 drives the air to flow inside the mask and the hood.

Specifically, the air enters the mask 10 and the hood 30 through the air inlet 13, the air inside the mask 10 and the hood 30 is guided by the air guide 50 to the air outlet 12, and then discharged to the outside of the mask 10 and the hood 30 through the air outlet 12. The barrier layer 40 is for blocking external air from entering through the connection between the mask 10 and the hood 30 into the inside of the mask 10 and the hood 30. In the present embodiment, the number of the air inlets 13 and the number of the filters 20 are both two, with each air inlet 12 disposed with a filter 20.

It should be noted that in the present embodiment, as shown in FIG. 2, the protective mask 100 requires a protective clothing 200 to be worn at the same time, so as to cover the whole body and further achieve the full-body blocking effect. However, the present invention is not limited to this restriction. In another preferred embodiment not shown in the figure, the protective mask 100 and the protective clothing 200 can be integrally formed.

To provide a further understanding of the structural characteristics of the present invention, the use of technical means and the expected effects, the method of use of the present invention is described to further a deeper and specific understanding of the present invention, as follows:

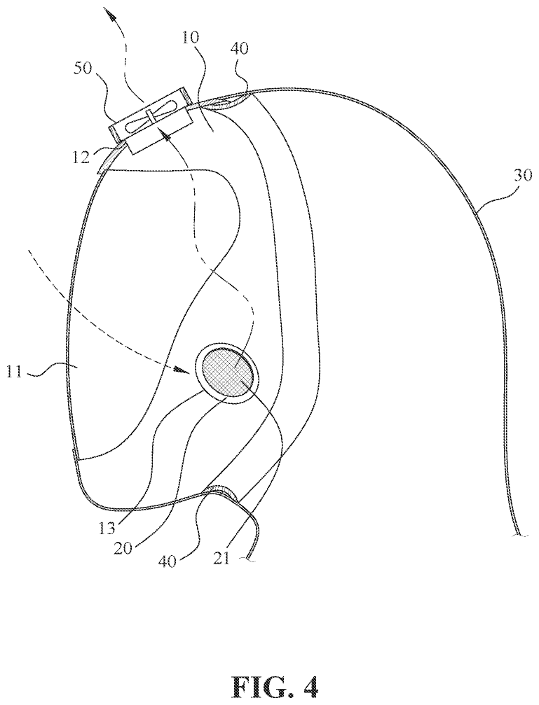

Referring to FIG. 4, in combination with FIGS. 1-3, FIG. 4 is a schematic view of the use state of the first embodiment of the present invention. the air enters the mask 10 and the hood 30 through the air inlet 13, the air inside the mask 10 and the hood 30 is guided by the air guide 50 to the air outlet 12, and then discharged to the outside of the mask 10 and the hood 30 through the air outlet 12. The barrier layer 40 is for blocking external air from entering through the connection between the mask 10 and the hood 30 into the inside of the mask 10 and the hood 30.

It should be noted that, the air enters the mask 10 and the hood 30 through the air inlet 13. At the same time, the filtering material 21 of the filter 20 filters the airborne droplets, pollutants, pathogens, pollen, floating fine asbestos fiber dust and other risk factors in the air, and the filtered air enters the inside of the mask 10 and the hood 30; then, the filtered air is discharged to the outside of the mask 10 and the hood 30 through the air outlet 12. The air guide 50 drives the air inside the mask 10 and the hood 30 to flow, so that the filtered air is guided to the air outlet 12, and the air inside the mask 10 and the hood 30 is vented to the outside through the air outlet 12, so as to reduce the stuffiness inside protective clothing. At the same time, the staff can wear the protective mask 100 for filtering the airborne droplets, pollutants, pathogens, pollen, floating fine asbestos fiber dust and other risk factors to increase the safety of use.

As such, the protective mask 100 provided by the present invention is mainly by the use of the arrangement of air outlet 12 and air inlet 13, and with the arrangement of filters 20 and air guides 50. The air enters the inside of the mask 10 and the hood 30 through the air inlet 13, the air guide 50 is used to guide the air inside the mask 10 and the hood 30 to the air outlet 12, and the air is discharged to the outside of the mask 10 and the hood 30 through the air outlet 12. The barrier layer 40 is used to block the outside air from entering the inside of the mask 10 and the hood 30 through the joint between the mask 10 and the hood 30 to block the external polluted air, impurities and dust, so that the present invention can reduce the stuffiness inside the protective clothing and further improve the work efficiency, as well as greatly increase the work time the wearer can withstand. Furthermore, the present invention can filter the airborne droplets, pollutants, pathogens, pollen, floating fine asbestos fiber dust and other risk factors to increase the safety of use.

### Second Embodiment

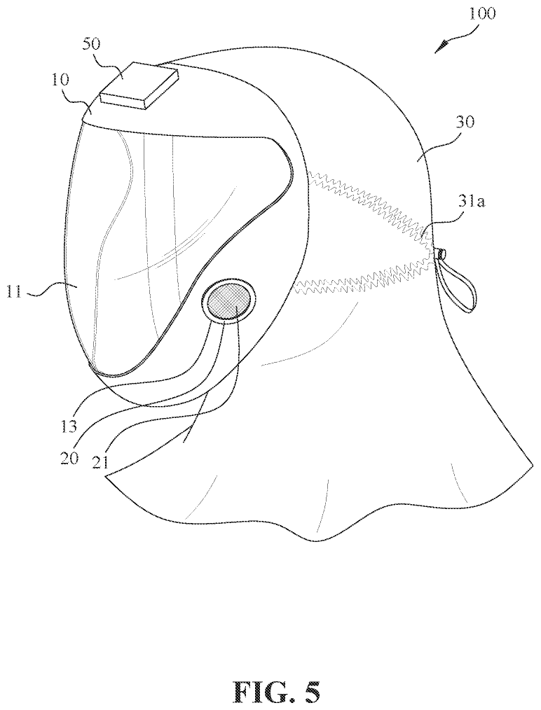

Referring to FIG. 5. FIG. 5 is a perspective view of the second embodiment of the present invention. Compared to the first embodiment, the second embodiment differs in that the back of the hood 30 is provided with at least one adjustable element 31a, and the adjustable element 31a is used to adjust the tightness of the hood 30.

Specifically, to conform to the head shapes of different workers, when the worker wears the protective mask 100, the hood 30 conforms to the head shape of the worker by adjusting the adjustable member 31a, and the hood 30 is flexible.

As such, the second embodiment can not only achieve the effects of the first embodiment, but also provide different structures. The second embodiment can be configured with the adjustable element 31a so that the protective mask 100 can match different head shape of the worker by adjusting the tightness of the hood 30 to increase the applicability and convenience of the protective mask 100.

### Third Embodiment

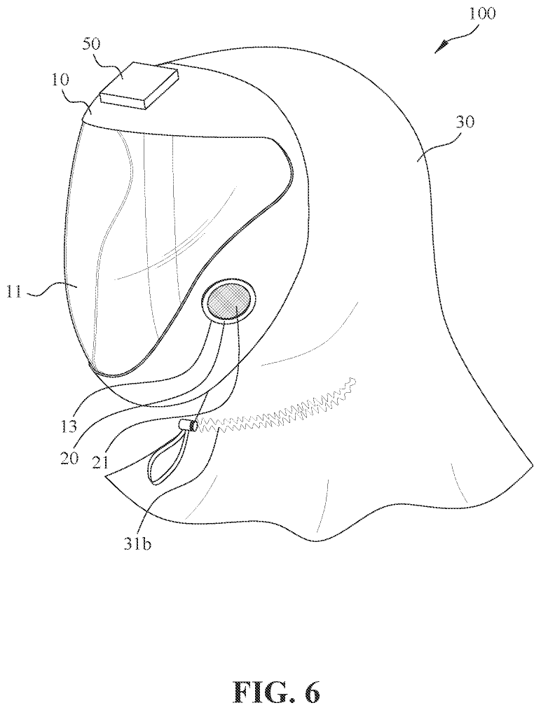

Referring to FIG. 6, FIG. 6 is a perspective view of the third embodiment of the present invention. Compared to the first embodiment and the second, the third embodiment differs in that the chin portion of the hood 30 is provided with at least one adjustable element 31b, and the adjustable element 31b is used to adjust the tightness of the hood 30.

To conform to the face shapes of different workers, when the worker wears the protective mask 100, the hood 30 conforms to the face shape and chin shape of the worker by adjusting the adjustable element 31b. The present invention is not limited to this. In another preferred embodiment not shown in the figure, the back of the head cover 30 and the chin portion are respectively provided with the adjustable elements 31a, 31b, so that the protective mask 100 can be adjusted to correspond to different head, face and chin shapes at the same time.

As such, the third embodiment can not only achieve the effects of the first and second embodiments, but also provide different structures. The protective mask 100 of the third embodiment can adjust the hood 30 through the arrangement of the adjustable element 31b to fit the face shape and chin shape of different workers, thereby increasing the applicability and convenience of the protective mask 100.

### Fourth Embodiment

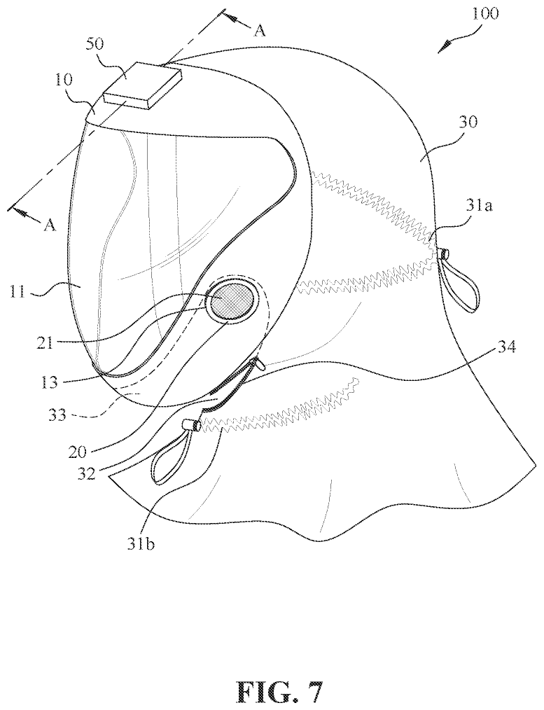

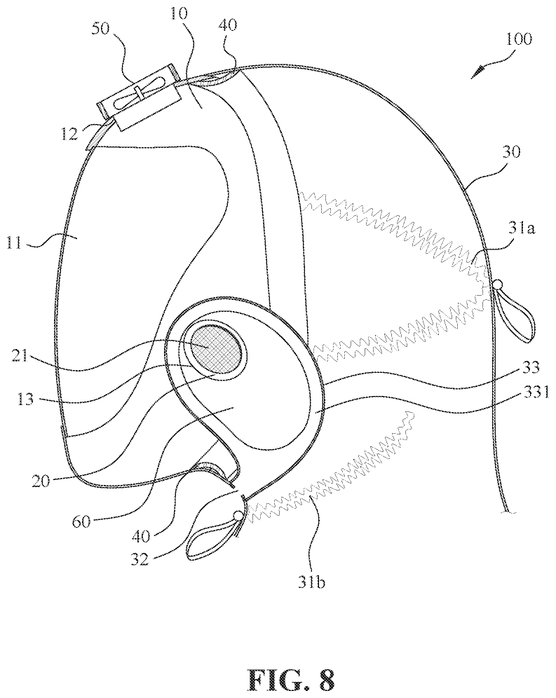

Referring to FIG. 7 and FIG. 8, as well as FIG. 4, FIG. 7 is a perspective view of the fourth embodiment of the present invention; FIG. 8 is a schematic cross-sectional view taken along line A-A in FIG. 7. Compared with the first, second, and third embodiments, the fourth embodiment differs in that the chin portion of the head cover 30 is disposed with an opening 32 in a recessed manner, and the opening 32 is connected to an inner bag 33. The inner bag 33 has an accommodating space 331, the inner bag 33 is located inside the mask 10 and the hood 30, and the inner bag 33 covers the air inlet 13 and the opening 32. A zipper 34 is arranged at the edge of the inner bag 33, and the zipper 34 is used to open or close the opening 32, and further includes at least one filtering material 60 arranged in the accommodating space 331 of the inner bag 33.

It should be noted that the material of the inner bag 33 is selected from one or a combination of cooling, antibacterial and skin-friendly materials. The air enters the mask 10 and the hood 30 through the air inlet 13, and the filtering material 21 and the filtering material 60 filter the airborne droplets, pollutants, pathogens, pollen, floating fine asbestos fiber dust and other risk factors, and the filtered air enters the inside of the mask 10 and the hood 30. Then, the filtered air is discharged to the outside of the mask 10 and the hood 30 through the air outlet 12, while the air guide 50 drives the air flow inside the mask 10 and the hood 30. The filtered air is guided to the air outlet 12, and the air inside the mask 10 and the hood 30 is vented to the outside through the air outlet 12, so as to reduce the stuffiness inside the protective clothing. When the staff wears the protective mask 100, the airborne droplets, pollutants, pathogens, pollen, floating fine asbestos fiber dust and other risk factors are filtered, so as to increase the safety of use.

Therefore, the fourth embodiment can not only achieve the effects of the first, second, and third embodiments, but also provide different structures. The protective mask 100 of the fourth embodiment performs a second filtering on the inhaled air through the use of the opening 32, the inner bag 33, the zipper 34 and the filtering material 60, thereby further enhancing the safety of the staff.

Hereby, the characteristics of the present invention and expected effects are stated as follows:

The protective mask 100 provided by the present invention is mainly by the use of the arrangement of air outlet 12 and air inlet 13, and with the arrangement of filters 20 and air guides 50. The air enters the inside of the mask 10 and the hood 30 through the air inlet 13, the air guide 50 is used to guide the air inside the mask 10 and the hood 30 to the air outlet 12, and the air is discharged to the outside of the mask 10 and the hood 30 through the air outlet 12. The barrier layer 40 is used to block the outside air from entering the inside of the mask 10 and the hood 30 through the joint between the mask 10 and the hood 30 to block the external polluted air, impurities and dust, so that the present invention can reduce the stuffiness inside the protective clothing and further improve the work efficiency, as well as greatly increase the work time the wearer can withstand. Furthermore, the present invention can filter the airborne droplets, pollutants, pathogens, pollen, floating fine asbestos fiber dust and other risk factors to increase the safety of use.

As such, the present invention has the following implementation and technical effects:

First, through the arrangement of the air inlet 13 and the filter 20, the protective mask 100 of the present invention can filter airborne droplets, pollutants, pathogens, pollen, and floating fine asbestos fiber dust in the air and other risk factors, so as to enhance the safety of the staff.

Second, the present invention utilizes the disposition of air outlet 12 and the air guide 50, the air guide 50 drives the air to flow inside the mask 10 and the hood 30 to the air outlet 12, and the filtered air inside the mask 10 and the hood 30 is vented through the air outlet 12 to the outside, so that the protective mask 100 can reduce the stuffiness in the protective clothing.

Third, the present invention utilizes the disposition of the barrier layer 40, so that the protective mask 100 blocks the external air from entering the mask 10 and the hood 30 through the connection between the mask 10 and the hood 30.

Fourth, the protective mask 100 of the present invention includes no excessive components, so as to reduce the overall weight of the protective mask 100, so as to reduce the pressure on the user's head.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A protective mask, comprising:
a mask, comprising at least a pair of protective goggles, at least one air outlet and at least one air inlet, the air outlet being disposed at the upper end of the protective goggles, the air inlet being disposed at the lower end of the protective goggles, the air inlet being disposed with a filter, both the air inlet and the air outlet communicating with the inside of the mask;
a hood, connected to the mask, and covering the edge of the mask;
at least one barrier layer, disposed at the connection between the mask and the hood; and
at least one air guide, disposed at the air outlet;
wherein air entering the mask and the hood through the air inlet, the air inside the mask and the hood being guided by the air guide to the air outlet, and then discharged to the outside of the mask and the hood through the air outlet, and the barrier layer blocking external air from entering through the connection between the mask and the hood into the mask and the hood.

2. The protective mask according to claim 1, wherein the filter comprises a filtering material for filtering harmful substances in the air.

3. The protective mask according to claim 1, wherein the air guide is a fan, and the air guide drives the air to flow inside the mask and the hood.

4. The protective mask according to claim 1, wherein the number of the air inlets and the number of the filters are both two, with each air inlet disposed with a filter.

5. The protective mask according to claim 1, wherein the back of the hood is provided with at least one adjustable element, and the adjustable element is used to adjust the tightness of the hood.

6. The protective mask according to claim 1, wherein the chin portion of the hood is provided with at least one adjustable element, and the adjustable element is used to adjust the tightness of the hood.

7. The protective mask according to claim 1, wherein an opening is disposed in the chin portion of the hood in a recessed manner, and the opening is connected to an inner bag, and the inner bag has an accommodating space.

8. The protective mask according to claim 7, wherein the inner bag is located inside the mask and the hood, and the inner bag covers the air inlet.

9. The protective mask according to claim 7, wherein a zipper is provided at the edge of the opening, and the zipper is used to open or close the opening.

10. The protective mask according to claim 8, wherein at least one filtering material is further disposed in the accommodating space of the inner bag.
